(19) United States
(12) Patent Application Publication
(10) Pub. No.: US 2009/0171141 A1
(43) Pub. Date: Jul. 2, 2009

(54) Anterior Repair - Needle Path and Incision Sites

(76) Inventor: Michael S. H. Chu, Brookline, MA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/341,540

(22) Filed: Dec. 22, 2008

Related U.S. Application Data
(60) Provisional application No. 61/016,984, filed on Dec. 27, 2007.

(51) Int. Cl. A61B 17/06; A61F 2/00; A61B 19/00

(52) U.S. Cl. 600/37; 606/222; 128/898

(57) ABSTRACT

In one embodiment, a medical device includes a support portion having a first end portion and a second end portion opposite the first end portion. An elongate member extends from the first end portion of the support portion and has a length sufficient to extend from the first end portion of the support portion to a location above a pubic bone when the support portion is disposed between the bladder and a vagina of the patient. An elongate member extends from the second end portion of the support portion and has a length sufficient to extend from the second end portion of the support portion and through an obturator membrane of the patient when the support portion is disposed between the bladder and the vagina of the patient.

### CROSS-REFERENCE TO RELATED APPLICATION

This application claims priority to U.S. Provisional Patent Application No. 61/016,984, filed Dec. 27, 2007, entitled “Anterior Repair—Needle Path and Incision Sites,” the entire content of which is hereby incorporated by reference.

### BACKGROUND

This invention relates to a medical device and more particularly to a medical device configured to be placed within a pelvic floor region of a patient.

The disclosed embodiments have application in a variety of surgical procedures relating to the female pelvic floor. For example, one such procedure is directed to insertion of a support device into a pelvic floor region of a patient to support an anatomical structure. The pelvic floor wall of women may weaken as a result of childbirth or menopause. In some of these cases, the wall tears and results in, for example, a cystocele or a hernia of the bladder.

Thus, a need exists for a support device that can help support a bladder or other anatomical structure disposed within a pelvic region of a patient.

### SUMMARY OF THE INVENTION

In one embodiment, a medical device includes a support portion having a first end portion and a second end portion opposite the first end portion. An elongate member extends from the first end portion of the support portion and has a length sufficient to extend from the first end portion of the support portion to a location above a pubic bone when the support portion is disposed between the bladder and a vagina of the patient. An elongate member extends from the second end portion of the support portion and has a length sufficient to extend from the second end portion of the support portion and through an obturator membrane of the patient when the support portion is disposed between the bladder and the vagina of the patient.

In another embodiment, a method is directed to placing a medical device within a body of a patient. The medical device has a support portion. A first extension portion has a elongate member that extends from a first end portion of the support portion and a second extension portion has a elongate member that extends from a second end portion of the support portion. The medical device is placed within a pelvic region of the patient such that the support portion is disposed between a bladder and a vagina of the patient. An end portion of the elongate member of the first extension portion is extended to a location above a pubic bone of the patient. An end portion of the elongate member of the second extension portion is extended through an obturator membrane of the patient.

### BRIEF DESCRIPTION OF THE DRAWINGS

The present invention is described with reference to the accompanying drawings.

FIG. 1 is a schematic illustration of an embodiment of a support device.

FIG. 2 is a top view of an embodiment of a support device.

FIG. 3 is a front view of a support device shown disposed within the pelvic region of a patient.

FIG. 4 is a side perspective view of the support device of FIG. 4 disposed within the pelvic region of a patient.

FIG. 5 is a flowchart illustrating a method of placing an embodiment of the medical support device within a body of a patient.

FIG. 6 is a top view of another embodiment of a support device.

### DETAILED DESCRIPTION

The medical devices described herein can be inserted and placed into a body of a patient. For example, the medical support device can be placed within a body of a patient to support an anatomical structure such as a bladder. Specifically, the medical support device can have elongated arms to help retain the support device at a location with respect to the anatomical structure to be supported. The elongated arms can be placed within muscle or other bodily tissue to anchor the arms with respect to the muscle.

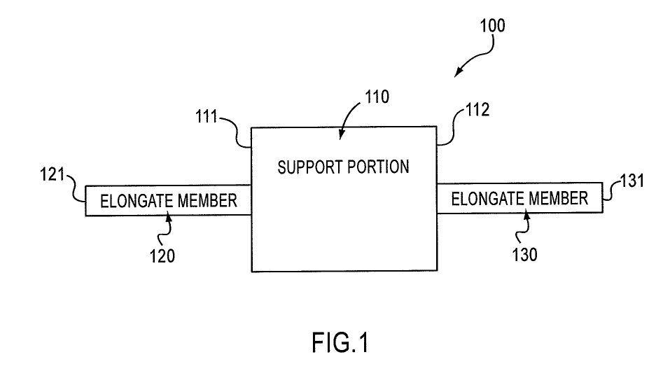

FIG. 1 is a schematic illustration of an embodiment of a support device. An apparatus 100 (also referred to herein as “support device”) can be placed or otherwise inserted into a body of a patient, such as, for example, within a pelvic floor region of the body.

The support device 100 includes a support portion 110 having a first side portion 111 and a second side portion 112 opposite the first side portion 111. The support portion 110 has multiple elongated members 120 and 130 extending from the support portion 110. Specifically, the elongate member 120 extends from the first side portion 111 of the support portion 110, and the elongated member 130 extends from the second side portion 112 of the support portion 110.

The elongate members 120 and 130 each have a length sufficient to help anchor the support device 100 with respect to tissue or anatomical structures in a pelvic region of a patient. Specifically, in one embodiment, the elongate member 120 of the first side portion 111 has a length sufficient to extend from the first side portion 111 of the support portion 110 to a location above a pubic bone of the patient when the support portion 110 is disposed between a bladder and a vagina of the patient. The elongate member 130 of the second side portion 112 has a length sufficient to extend from the second side portion 112 of the support portion 110 and through an obturator membrane of the patient when the support portion 110 is disposed between the bladder and the vagina of the patient.

The end portions 121 and 131 of the elongate members 120 and 130, respectively, can be coupled within muscle of the patient to anchor the support device 100 with respect to the pelvic region of the patient. Specifically, the end portion 121 of the elongate member 120 of the first side portion 111 can be coupled to muscle or other tissue located at or near the suprapublic location of the patient. The end portion 131 of the elongate member 130 of the second side portion 112 can be coupled to muscle or other tissue located at or near the obturator membrane of the patient. In some embodiments, the end portions 121 and 131 of the elongate members 120 and 130, respectively, can extend outside of the patient. Thus, the elongate member 120 and 130 can be used to help externally adjust the support device 100 with respect to tissue or anatomical structure in the pelvic region of the patient. Specifically, the elongate members 120 and 130 can be moved via a force applied to the elongate members 120 and 130 external to the patient, which thereby can move the support portion 110 of the support device 100 within the pelvic region of the patient. In some embodiments, the end portions 121 and 131 can be coupled to the skin of the patient.

The support device 100 can support an anatomical structure or tissue, such as, for example, a bladder, a uterus, a urethra, or the like.

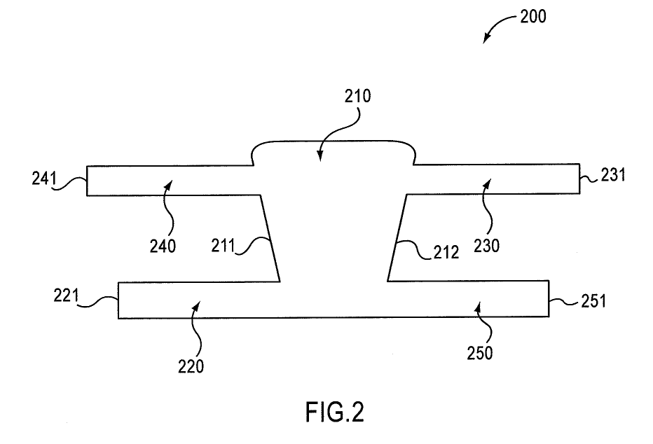

FIG. 2 is a top view of an embodiment of a support device. The support device 200 includes a support portion 210 and multiple elongate members 220, 230, 240 and 250 each extending from the support portion 210. The support portion 210 is configured to help support a tissue or an anatomical structure (e.g., a bladder, a uterus, a urethra) located in the pelvic region of the patient.

The support portion 210 has a first side portion 211 and a second side portion 212 opposite the first side portion 211. A first elongate member 220 and a second elongate member 240 extend from the first side portion 211 of the support portion 210. A first elongate member 250 and a second elongate member 230 extend from the second side portion 212 of the support portion 210. Each of the elongate members 220, 230, 240 and 250 is configured to help retain the support portion 210 at a location in the pelvic region of a patient with respect to the anatomical structure that is being supported.

The first elongate member 220 of the first side portion 211 and the first elongate member 250 of the second side portion 212 can be disposed, in part, external to the patient. Thus, the support portion 210 can be adjusted within the patient via an external force applied to the first elongate member 220 of the first side portion 211 and/or the first elongate member 250 of the second side portion 212 as described above. Specifically, the first elongate member 220 of the first side portion 211 has a length sufficient to extend from the first side portion 211 of the support portion 210 and through a skin incision at or near a first location above a pubic bone. The first elongate member 250 of the second side portion 212 has a length sufficient to extend from the second side portion 212 of the support member 210 and through a skin incision at or near a second location above a pubic bone different from the first location above the pubic bone.

In this embodiment, the first elongate member 220 of the first side portion 211 and first elongate member 250 of the second side portion 212 can be anchored within the patient at or near the abdominal region of the patient. Specifically, muscle and/or skin at the first location above the pubic bone surrounds and maintains the position of the first elongate member 220 of the first side portion 211. Similarly, muscle, skin and/or other bodily tissue at the second location above the pubic bone surrounds and maintains the positions of the first elongate member 250 of the second side portion 212.

In an alternative embodiment, an end portion of the first elongate member of the first side portion and an end portion of the first elongate member of the second side portion do not extend external to the patient and instead are disposed within the first and second locations above the pubic bone, respectively. Specifically, the first elongate member of the first side portion has a length sufficient to extend from the first side portion of the support portion to the first location above the pubic bone when the support portion is disposed between a bladder and a vagina of the patient. The first elongate member of the second side portion has a length sufficient to extend from the second side portion of the support portion to the second location above the pubic bone when the support portion is disposed between the bladder and the vagina of the patient.

In the illustrated embodiment, the second elongate member 240 of the first side portion 211 and the second elongate member 230 of the second side portion 212 can be disposed, in part, external to the patient. Thus, the support portion 210 can be adjusted within the patient via an external force applied to the second elongate member 220 of the first side portion 211 and/or the second elongate member 230 of the second side portion 212 as described above. Specifically, the second elongate member 240 of the first side portion 211 has a length sufficient to extend from the first side portion 211 of the support portion 210 through a first obturator membrane of the patient and through a skin incision near or adjacent to a first obturator foramen of the patient. The second elongate member 230 of the second side portion 212 has a length sufficient to extend from the second side portion 212 of the support portion 210 through a second obturator membrane of the patient and through a skin incision near or adjacent to a second obturator foramen of the patient.

In one embodiment, the second elongate member 240 of the first side portion 211 and the second elongate member 230 of the second side portion 212 can extend through one or more muscles. Specifically, the second elongate member 240 of the first side portion 211 has a length sufficient to extend from the support portion 210 through a levator ani muscle and through the first obturator membrane. The second elongate member 230 of the second side portion 212 has a length sufficient to extend from the support portion 210 through a levator ani muscle and through the second obturator membrane. The second elongate member 240 of the first side portion 211 and/or the second elongate member 230 of the second side portion 212 can extend through the tendinous arch, iliococcygeus, pubococcygeus, and/or puborectalis muscle, which all are part of the levator ani muscle.

The second elongate member 240 of the first side portion 211 and the second elongate member 230 of the second side portion 212 can be anchored within the patient at or near the buttock region of the patient. Specifically, muscle and/or skin near the first obturator foramen, first levator ani muscle and/or the first obturator membrane surrounds and maintains the position of the second elongate member 240 of the first side portion 211. Similarly, muscle, skin and/or other bodily tissue near the second obturator foramen, the second levator ani muscle and/or the second obturator membrane surrounds and maintains the positions of the second elongate member 230 of the second side portion 212.

In an alternative embodiment, an end portion of the second elongate member of the first side portion and an end portion of the second elongate member of the second side portion do not extend external to the patient. Instead, the end portions of each of the elongate members are disposed within the body of the patient. For example, in one embodiment, the second elongate member of the first side portion has a length sufficient to extend from the first side portion of the support portion and through the first obturator membrane of the patient when the support portion is disposed between the bladder and the vagina of the patient. The second elongate member of the second side portion has a length sufficient to extend from the second side portion of the support portion and through the second obturator membrane of the patient when the support portion is disposed between the bladder and the vagina of the patient.

Although the term “side portion” of the support portion is used herein, it should be understood that the “side portion” of the support portion can also be referred to as an end portion of the support portion.

In the illustrated embodiment, the support portion 210 is shown in FIG. 2 as having a trapezoidal shape. It should be understood that the support portion can have any shape, including for example, a rectangular shape, a triangular shape, a square shape and an ovular shape.

In some embodiments, the elongate members of the support device can be placed at different angles with respect to the support portion. In some embodiments, the elongate members can extend from various locations of the support portion. In some embodiments, each of the elongate members can be placed in different body portions of the patient, such as, for example, sacrospinous ligament, ischio-coccygeus muscle, etc.

In some embodiments, the elongate members of the support device can include tangs, nubs, dimples and/or tabs that are configured help anchor the support device with respect to tissue. The tangs, nubs, dimples, and/or tabs, for example, can engage tissue upon removal of a delivery sleeve from the elongate members. In some embodiments, the elongate members of the support device can include sutures to help anchor the support device to tissue.

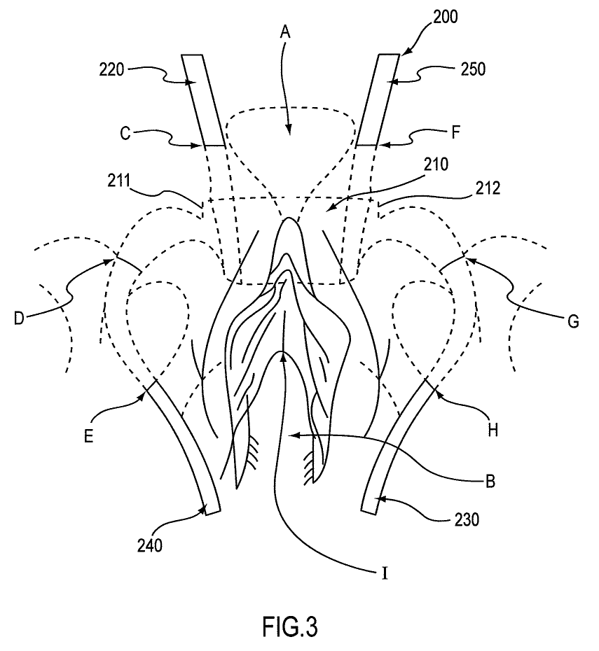

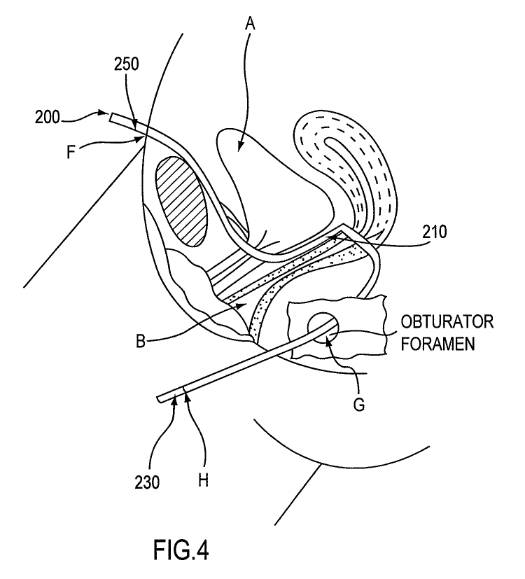

FIGS. 3 and 4 are a front view and a side perspective view, respectively, of the support device 200 shown positioned within the pelvic region of a patient. The support device 200 is shown disposed between a bladder A and a vagina B. As discussed above, each elongate member 220, 230, 240 and 250 are anchored within muscle or other tissue while anchoring the support portion 210 between them to provide support for an anatomical structure, such as, for example, the bladder A, a uterus or the like. In other words, the support portion 210 is retained at a fixed position with a fixed tension and can help support the bladder A. The support device 200 is inserted into the patient through a vaginal incision I. The first elongate member 220 extends from the first side portion 211 of a support portion 210 of the support device 200 through a skin incision C at a first location above a pubic bone. A second elongate member 240 extends from the first side portion 211 of the support portion 210 and through both a first obturator membrane D and a skin incision E at or near the first obturator membrane D. A first elongate member 250 extends from the second side portion 212 of the support portion 210 and through a skin incision F at a second location above the pubic bone. A second elongate member 230 extends from the second side portion 212 of the support portion 210 and through both a second obturator membrane G and a skin incision H at or near a second obturator membrane G.

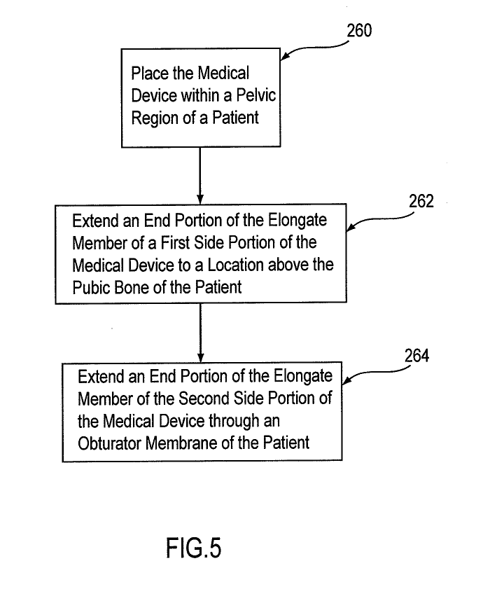

FIG. 5 is a flowchart illustrating a method of placing an embodiment of the support device 200. At 260, the support device 200 is placed within a pelvic region of the patient such that the support portion 210 is disposed between a bladder and a vagina of the patient. At 262, an end portion 221 of the first elongate member 220 of the first side portion 211 is extended to a first location above a pubic bone of the patient. At 264, an end portion 231 of the second elongate member 230 of the second side portion 212 is extended through a first obturator membrane of the patient.

In one embodiment, five skin incisions are made to the patient to place and anchor the support device 200 within a body of a patient. Specifically, a vaginal incision, a skin incision at a first location above the pubic bone, a skin incision at a second location above the pubic bone, a skin incision near or adjacent to a first obturator foramen and a skin incision near or adjacent to a second obturator foramen are made to the patient.

The support device 200 is inserted into the pelvic region through the vaginal incision before the placing of the support device 200. The order of placement of the elongate members of the support device 200 is at the discretion of the user. In one embodiment, the end portion 221 of the first elongate member 220 of the first side portion 211 and the end portion 251 of the first elongate member 250 of the second side portion 212 are extended through the skin incision at the first location above the pubic bone and the skin incision at the second location above the pubic bone, respectively.

The end portion 241 of the second elongate member 240 of the first side portion 211 and the end portion 231 of the second elongate member 230 of the second side portion 212 are extended through the skin incision near or adjacent to the first obturator foramen and the skin incision at the second obturator foramen, respectively. In some embodiments, a levator ani muscle incision is made to the patient near the ischial spine. The end portion of the second elongate member of the first side portion is extended through the levator ani muscle incision, through the obturator foramen and through the skin incision near or adjacent to the first obturator foramen. The second elongate member of the second side portion can be extended through an incision in another levator ani muscle as described above.

An insertion device (not shown) can be used to insert the support device 200 into the patient. The insertion device can be one or more needles configured to facilitate insertion through tissue and move the first elongate member 220 of the first side portion 211 of the support portion 210 of the support device 200 through the tissue. A first needle is coupled to the support device 200 after insertion of the support device through the vaginal incision. Specifically, the first needle is inserted into the body through a skin incision at the first location above the pubic bone to create a first passageway, and then coupled to the first elongate member 220 of the first side portion 211. The first needle while coupled to the first elongate member 220 of the first side portion 211 can then be withdrawn to dispose the first elongate member 220 of the first side portion 211 within the first passageway such that the end portion 221 of the first elongate member 220 of the first side portion 211 extends from the skin incision at the first location above the pubic bone. Similarly, a second needle can be inserted into the body through a skin incision at a second location above the pubic bone to create a second passageway, and then coupled to the first elongate member 250 of the second side portion 212. The second needle while coupled to the first elongate member 250 of the second side portion 212 can then be withdrawn to dispose the first elongate member 250 of the second side portion 212 within the second passageway such that the end portion 251 of the first elongate member 250 of the second side portion 212 extends from the skin incision at the second location above the pubic bone.

A third needle and a fourth needle can be used to extend the second elongate member 230 and 240. Specifically, the third needle can be inserted into the body through a skin incision near or adjacent to a first obturator foramen to create a third passageway, and then coupled to the second elongate member 240 of the first side portion 211. The third needle while coupled to the second elongate member 240 of the first side portion 211 can then be withdrawn to dispose the second elongate member 240 of the first side portion 211 within the third passageway such that the end portion 241 of the second elongate member 240 of the first side portion 211 extends from the skin incision near or adjacent to the first obturator foramen. Similarly, the fourth needle can be inserted into the body through a skin incision near or adjacent to a second obturator foramen to create a fourth passageway, and then coupled to the second elongate member 230 of the second side portion 212. The fourth needle while coupled to the second elongate member 230 of the second side portion 212 can then be withdrawn to dispose the second elongate member 230 of the second side portion 212 within the fourth passageway such that the end portion 231 of the second elongate member 230 of the second side portion 212 extends from the skin incision near or adjacent to the second obturator foramen.

Alternatively, each needle can be coupled to the end portion of an elongate member of the support device prior to insertion of the support device into the patient. Each needle can then be passed through a vaginal incision, and through a skin incision at a location above the pubic bone or through a skin incision at or near an obturator foramen.

The insertion device can include a dilator and connectors to facilitate passage of the elongate members through tissue. Specifically, the needle can be coupled to the dilator via the connector. The dilator has a width greater than a width of the needle such that the dilator increases a width of the passageway before passage of an elongate member. In another embodiment, the dilator is equal to or less than the width of the needle to minimize the passageway created. The insertion device can also include markings and/or colors. In an alternative embodiment, a pusher be used in place of the needle.

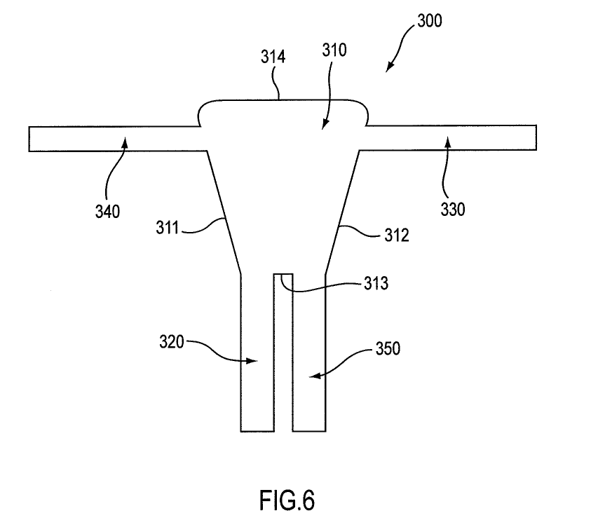

FIG. 6 is a top view of an embodiment of a support device. The support device 300 includes a support portion 310 having multiple side portions 311, 312, 313 and 314. Specifically, the support portion 310 has a first side portion 311 and a second side portion 312 opposite the first side portion 311. The support portion 310 also has a third side portion 313 and a fourth side portion 314 opposite the third side portion 313. A first elongate member 320 and a second elongate member 350 extend from the third side portion 313. An elongate member 340 extends from the first side portion 311. An elongate member 330 extends from the second side portion 312.

The first and second elongate members 320 and 350 of the third side portion 313 each have a length sufficient to extend from the third side portion 313 of the support portion 310 through a skin incision at a first location and a second location above the pubic bone, respectively, when the support portion 310 is disposed between the bladder and the vagina of the patient as described above.

The elongate members 340 and 330 of the first side portion 311 and the second side portion 312 each have a length sufficient to extend from the support portion 310 and respectively through a first and a second obturator membrane and a skin incision near a first and a second obturator membrane of the patient.

In another embodiment, a support device has a support portion with a first side portion and a second side portion opposite the first side portion. An elongate member extends from the first side portion of the support portion. An elongate member extends from the second side portion of the support portion. The elongate member of the first side portion has a length sufficient to extend from the first side portion of the support portion through a skin incision at a first location above a pubic bone. Similarly, the elongate member of the second side portion has a length sufficient to extend from the second side portion of the support portion through a skin incision at a second location above a pubic bone. At least a portion of the support portion is secured or anchored to tissue via for example, stitches, sutures or the like. Stitches can be placed via a stitching device (e.g., a capio device). For example, the stitches can be used to couple a fourth side portion of the support portion to bodily tissue.

In yet another alternative embodiment, a support device (not shown) has a support portion with a first side portion and a second side portion opposite the first side portion. An elongate member extends from the first side portion of the support portion. An elongate member extends from the second side portion of the support portion. The elongate member of the first side portion has a length sufficient to extend from the first side portion of the support portion through a skin incision at or near a first obturator foramen. Similarly, the elongate member of the second side portion has a length sufficient to extend from the second side portion of the support portion through a skin incision at or near a second obturator foramen. At least a portion of the support portion is secured or anchored to tissue via for example, stitches, sutures or the like. Stitches can be placed via a stitching device (e.g., a capio device).

In yet still another embodiment, a soft tissue anchor or dilator can be associated to the elongate members of the support device such that only a single vaginal incision is required (without any other skin incisions). The elongate members have a length that is not sufficient to extend through the skin. In this embodiment, the soft tissue anchor or dilator is delivered with a delivery device from inside the vaginal incision, deeper into tissue such as the levator ani muscle and not towards, nor through the obturator. The other elongate member is placed towards the skin above a pubic bone, but not through the skin above the pubic bone.

The support device can be formed from any material or materials known in the art to be used in constructing support devices. For example, biocompatible polymers can be used in producing the support portion of the support device. In some embodiments, the support device can include a shape memory material, such as, for example, nitinol, etc.

In one embodiment, a support portion has a first end portion and a second end portion opposite the first end portion. An elongate member extends from the first end portion of the support portion and has a length sufficient to extend from the first end portion of the support portion to a location above a pubic bone when the support portion is disposed between the bladder and a vagina of the patient. An elongate member extends from the second end portion of the support portion and has a length sufficient to extend from the second end portion of the support portion and through an obturator membrane of the patient when the support portion is disposed between the bladder and the vagina of the patient.

In some embodiments, the support portion is configured to support a bladder of a patient or a uterus of the patient.

In some embodiments, the elongate member that extends from the first end portion is a first elongate member that extends from the first end portion. A second elongate member extends from the first end portion of the support portion and has a length sufficient to extend from the first end portion of the support portion and through the obturator membrane of the patient when the support portion is disposed between the bladder and the vagina of the patient.

In some embodiments, the elongate member that extends from the second end portion is a first elongate member that extends from the second end portion. The location above the pubic bone is a first location above the pubic bone. A second elongate member extends from the second end portion of the support portion and has a length sufficient to extend from the second end portion of the support portion to a second location above the pubic bone different from the first location above the pubic bone when the support portion is disposed between the bladder and the vagina of the patient.

In some embodiments, the elongate member of the first end portion has a length sufficient to extend from the first end portion of the support portion and through a skin incision at the location above the pubic bone.

In some embodiments, the elongate member of the second end portion has a length sufficient to extend from the second end portion of the support portion through the obturator membrane of the patient and through a skin incision.

In some embodiments, the elongate member of the first end portion has a length sufficient to extend from the first end portion of the support portion and through a skin incision at the location above the pubic bone. The elongate member of the second end portion has a length sufficient to extend from the second end portion of the support portion through the obturator membrane of the patient and through a skin incision near the obturator membrane.

In some embodiments, the elongate member of the second end portion has a length sufficient to extend from the support portion through a levator ani muscle and through the obturator membrane.

In another embodiment, a method of placing a medical device within a body of a patient is described. The medical device has a support portion, an elongate member extending from a first end portion of the support portion and an elongate member extending from a second end portion of the support portion. The method includes placing the medical device within a pelvic region of the patient such that the support portion is disposed between a bladder and a vagina of a patient. The method includes extending an end portion of the elongate member of the first end portion to a location above a pubic bone of the patient and extending an end portion of the elongate member of the second end portion through an obturator membrane of the patient.

In some embodiments, the method includes producing a vaginal incision and inserting the medical device through the vaginal incision before the placing.

In some embodiments, the method includes producing an incision at the location above the pubic bone and extending the end portion of the elongate member of the first end portion through the incision at the location above the pubic bone.

In some embodiments, the method includes producing an incision at or near an obturator membrane and extending the end portion of the elongate member of the second end portion through the incision at or near the obturator membrane.

In some embodiments, the method includes producing an incision at or near a obturator membrane. The method includes producing a levator ani muscle incision and extending the end portion of the elongate member of the second end portion through the levator ani muscle incision and through the incision at or near the obturator membrane.

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

The previous description of the embodiments is provided to enable any person skilled in the art to make and use the invention. While the invention has been particularly shown and described with reference to embodiments thereof, it will be understood by those skilled in the art that various changes in form and details may be made. For example, a support device can include various combinations and sub-combinations of the various embodiments described herein.

What is claimed is:

1. A medical device, comprising:
a support portion having a first end portion and a second end portion opposite the first end portion;
an elongate member extending from the first end portion of the support portion and having a length sufficient to extend from the first end portion of the support portion to a location above a pubic bone when the support portion is disposed between the bladder and a vagina of the patient; and
an elongate member extending from the second end portion of the support portion and having a length sufficient to extend from the second end portion of the support portion and through an obturator membrane of the patient when the support portion is disposed between the bladder and the vagina of the patient.

2. The medical device of claim 1, wherein the support portion is configured to support a bladder of a patient or a uterus of the patient.

3. The medical device of claim 1, wherein the elongate member extending from the first end portion is a first elongate member extending from the first end portion, the medical device further comprising:
a second elongate member extending from the first end portion of the support portion and having a length sufficient to extend from the first end portion of the support portion and through the obturator membrane of the patient when the support portion is disposed between the bladder and the vagina of the patient.

4. The medical device of claim 1, wherein the elongate member extending from the second end portion is a first elongate member extending from the second end portion, the location above the pubic bone is a first location above the pubic bone, the medical device further comprising:
a second elongate member extending from the second end portion of the support portion and having a length sufficient to extend from the second end portion of the support portion to a second location above the pubic bone different from the first location above the pubic bone when the support portion is disposed between the bladder and the vagina of the patient.

5. The medical device of claim 1, wherein the elongate member of the first end portion has a length sufficient to extend from the first end portion of the support portion and through a skin incision at the location above the pubic bone.

6. The medical device of claim 1, wherein the elongate member of the second end portion has a length sufficient to extend from the second end portion of the support portion through the obturator membrane of the patient and through a skin incision.

7. The medical device of claim 1, wherein
the elongate member of the first end portion has a length sufficient to extend from the first end portion of the support portion and through a skin incision at the location above the pubic bone; and
the elongate member of the second end portion has a length sufficient to extend from the second end portion of the support portion through the obturator membrane of the patient and through a skin incision near the obturator membrane.

8. The medical device of claim 1, wherein the elongate member of the second end portion has a length sufficient to extend from the support portion through a levator ani muscle and through the obturator membrane.

9. A method of placing a medical device within a body of a patient, the medical device having a support portion, an elongate member extending from a first end portion of the support portion and an elongate member extending from a second end portion of the support portion, comprising:
placing the medical device within a pelvic region of the patient such that the support portion is disposed between a bladder and a vagina of a patient;
extending an end portion of the elongate member of the first end portion to a location above a pubic bone of the patient; and
extending an end portion of the elongate member of the second end portion through an obturator membrane of the patient.

10. The method of claim 9, further comprising:
producing a vaginal incision; and
inserting the medical device through the vaginal incision before the placing.

11. The method of claim 9, further comprising:
producing an incision at the location above the pubic bone; and
extending the end portion of the elongate member of the first end portion through the incision at the location above the pubic bone.

12. The method of claim 9, further comprising:
producing an incision at or near an obturator membrane; and
extending the end portion of the elongate member of the second end portion through the incision at or near the obturator membrane.

13. The method of claim 9, further comprising:
producing an incision at or near a obturator membrane;
producing a levator ani muscle incision; and
extending the end portion of the elongate member of the second end portion through the levator ani muscle incision and through the incision at or near the obturator membrane.
